(19) United States
(12) Patent Application Publication
(10) Pub. No.: US 2024/0390596 A1
(43) Pub. Date: Nov. 28, 2024

(54) Electronic system for a medical device, medical device with such an electronic system, external device for communicating with the electronic system, method for exchanging data between the medical device and the external device and corre

(71) Applicant: Sanofi, PARIS (FR)

(72) Inventors: Stefan Alt, Frankfurt am Main (DE); Ronald Anthony Smith, Salir, Loule (PT); Paul Richard Draper, Warwick Warwickshire (GB); John Whitehouse, Harvington, Evesham (GB); Hester Jane Corne, Warwick (GB)

(21) Appl. No.: 18/694,119

(22) PCT Filed: Sep. 22, 2022
(86) PCT No.: PCT/EP2022/076287

§ 371 Date: Mar. 21, 2024

(30) Foreign Application Priority Data
Sep. 24, 2021 (EP) 21315168.1

(51) Int. Cl. A61M 5/315; A61M 5/20; G16H 40/63; H04L 67/12

(52) U.S. Cl. CPC A61M 5/31568; A61M 5/20; G16H 40/63; A61M 2205/3379; A61M 2205/3561; H04L 67/12

(57) ABSTRACT

Electronic system for a medical device, medical device with such an electronic system, external device for communicating with the electronic system, method for exchanging data between the medical device and the external device and corresponding machine-readable code and data storage media comprising such code. Proposed is an electronic system for a medical device and for recording medical data, comprising an electronic control unit configured to transmit the medical data to an external device and/or store the medical data, wherein the electronic control unit (12) is configured to calculate a data integrity value (C) based on the medical data (B) and at least one additional data (A) not included in the medical data and wherein the electronic control unit is configured to, in correlation with the medical data, transmit the data integrity value to the external device and/or store the data integrity value.

Electronic system for a medical device, medical device with such an electronic system, external device for communicating with the electronic system, method for exchanging data between the medical device and the external device and corresponding machine-readable code and data storage media comprising such code

### BACKGROUND

Medical devices, especially injection devices (e.g. pen injectors or autoinjectors), blood pressure measurement devices, blood glucose level measurement devices, pulse measurement devices, electro-cardiogram devices and the like, are known to measure medical data and to record them. The recorded data is accessible via user interfaces on the medical devices. This leads to bulky medical devices, especially if those are handheld devices, and a high power consumption. Also, users often use smartphones to centralize their personal data.

Needle-based injection system (NIS) devices are used by patients to conveniently perform frequent (e.g. sub-cutaneous) injections, e.g. insulin for diabetics. These NIS devices usually have a mechanism to select the dose of the drug to be injected within certain limits, i.e. the dose to be injected may be varied within these limits. Recent NIS devices include electronics to measure and/or detect the selected and/or injected doses, and subsequently store it in an internal memory.

In small electromechanical medical devices, which are designed to record, store and possibly transmit diagnostic or therapy data (e.g. blood glucose level, dispensed insulin units, etc.), the embedded electronics impose limitations on the available memory space for these records.

### SUMMARY

An electronic system for a medical device (e.g. an injection device) and for recording medical data, e.g. medical data of or relating to the medical device, is proposed, the medical device comprising an electronic control unit configured to transmit the medical data to an external device and/or store to the medical data. This allows for sleek design of the medical device as the data may be viewed and administrated away from the external device. User interfaces on the medical device may be reduced, e.g. in number and/or functionality. This also allows for a lower power consumption as no user interfaces for displaying the collected data need to be powered. These data transmissions may encompass data about the most recent event performed by the medical device.

The electronic control unit may comprise a memory unit for storing the medical data. The memory unit may comprise a non-volatile memory to save on power consumption. The memory unit may for example comprise flash memory, or electrically erasable memory (NAND Flash memory, NOR Flash memory, EEPROM-electronically erasable programmable memory, NVRAM-non-volatile random access memory, etc.) or any other suitable memory. Alternatively the memory unit may comprise a volatile memory unit that needs a constant supply of power to upkeep the saved data. The electronic control unit and the memory unit may or may not be formed by a single integrated circuit. The electronic control unit may comprise an application-specific integrated circuit (ASIC). Alternatively the electronic control unit may comprise a processing unit for executing machine-readable code that is saved in the memory unit. The machine-readable code may be stored in a read-only memory of the memory unit. The machine-readable code may be stored in a rewritable memory to allow for updates.

Alternatively, the machine-readable code may be stored in the same memory as the medical data.

The electronic system may comprise a communication unit for transmitting data to the external device. “Transmitting data” herein may mean to provide instructions to the communication unit to transmit the data. The electronic control unit and the communication unit may or may not be formed by a single integrated circuit. The communication unit may be a wireless communication unit, e.g. based on Bluetooth Low Energy (BLE) technology, Bluetooth technology, NFC technology (near field communication), IEEE 802.11 (Wi-Fi) technology or other radio-transmission technology.

Alternatively, the communication unit may be a wire-based communication unit, e.g. based on universal serial bus (USB) technology, lightning technology or thunderbolt technology or other serial or parallel transmission technologies. The wire-based communication unit may comprise an (electromechanical) interface, e.g. a plug or a socket, to provide connectivity to the external device.

The external device may be a general purpose device, e.g. a PC or a Smartphone or the like, that is running dedicated machine-readable code to communicate with the electronic system, or a dedicated device, e.g. a special device with the sole purpose of communication with a single or multiple of the electronic systems and collecting the respective data. The external device may be a user terminal device. The external device may be a handheld device or a stationary device.

The electronic control unit being “configured to” perform a task herein may mean that it is specifically programmed and/or that it comprises specific circuitry to perform that task. During its operation, the control unit may perform the task.

According to further embodiments, the electronic control unit is configured to calculate a data integrity value based on the medical data and at least one additional data not included in the medical data and wherein the electronic control unit is configured to, in correlation with the medical data, transmit the data integrity value to the external device and/or to store the data integrity value. This allows for the detection of errors in the stored or transmitted medical data. Depending on the used method for calculation this may also allow for the correction of erroneous data. Additionally, as the additional data does not need to be transmitted or stored explicitly, this allows to reduce the amount of data needed to be saved or transmitted in correlation with the medical data. This allows the use of smaller memory units (memory units that store less data) and/or to record more data on the same memory unit. Also this may reduce power consumption. For safety reasons it is vital to include all required information in the medical data records to avoid potentially harmful ambiguities.

“Data” (e.g. additional data) may refer to multiple data or a single datum.

The data integrity value may be calculated according to a function that is sensitive at least to changing any single bit of the data used for the calculation. That a function is “sensitive” may mean that no matter what bit of the data used for calculation is flipped, it will give a result different from when doing the calculation with the original data or will give a unique result. The function may be sensitive to the change of any 2, 3, 4, 5, 6, 7, 8 or more bits of the data. The data integrity value may be calculated according to a hash-function, according to the cyclic redundancy check (CRC) method, or may be a checksum. The data integrity value may also be a signature type control value. An asymmetric encryption method may be used. When calculating the data integrity value, it may be encrypted using a private encryption key, e.g. known only to the electronic system, while the external device may use a corresponding public encryption key to decrypt the validation data. The medical data may also be encrypted (signed) by this method. That would also ensure data integrity, but additionally include cryptographic information (usually to validate the origin or creator of the data).

“Transmitting in correlation” herein may mean that the data integrity value and the medical data form a data package that is transmitted in a single transmission session, e.g. in a continuous data stream, or in multiple successive transmission sessions wherein a later transmission includes an identifier to reference an earlier data transmission for correlating the data integrity value and the respective medical data. In other words, the data integrity value and the medical data, after the transmission, may (still) be correlated.

“Storing in correlation” herein may mean that the data integrity value and the medical data form a data set. The medical data and the data integrity value may be stored in a fashion that represents a table. Multiple sets of medical data and corresponding data integrity values may be stored in a single table or table-like data structure.

According to another embodiment, the electronic control unit is configured to not transmit the additional data and/or it may be configured to not store the additional data in correlation with the medical data. The additional data may be saved or transmitted separately. The additional data may be transmitted, e.g. in other communication sessions or data-streams, preferably without reference to the data stream transmitting the medical data or vice versa. The additional data may be stored outside a data set or table comprising the medical data and the respective data integrity value. The additional data may be general data that is only saved or transmitted a single time, e.g. when pairing the medical device and the external device. In further cases, at least part of the additional data is never transmitted explicitly or individually to the external device. The additional data may be data that is already known to the external device and/or that can be estimated by the external device.

According to further embodiments, the additional data is or comprises at least one of a storage position identifier of the medical data, a device identifier, a device type identifier, a patient identifier, a therapy identifier, a medicament identifier, or a conversion factor identifier. The additional data may be deduced from other information (e.g. a continuous record number and/or storage position identifier). The additional data may be static (e.g. a patient identifier, type of medication, measurement unit, etc.). The additional data may be within a small value range (easily guessable).

The storage position identifier may be a record number identifying the position, e.g. column or row, of the medical data in a data structure, e.g. a table. Consecutively recorded medical data may be associated with consecutive integer values. The N-th recorded medical data may be stored in the N-th row of a table representation of a used data structure.

The device identifier may be a data value, e.g. an alpha-numerical string, to uniquely identify the device among all produced devices of the same type, of the same manufacturer and/or world-wide.

The device type identifier may be data that represents characteristics of the device, e.g. size, color, capacity and/or available features.

The patient identifier may be data representing a patients name or nickname or an associated number or alpha-numerical identifier. The patient identifier might even include a location or address information for a “lost and found” scenario.

The therapy identifier may be data representing a specific schedule for the use of the medical device. The electronic system might include an alarm unit to remind a user of the schedule.

The medicament identifier may be data representing a medicament or a type of medicament present in the medical device.

A conversion factor identifier may be data representing one or more units of measurement used for the medical data. The medical data may be stored and/or transmitted as a pure number value, e.g. as an integer, a floating point number or a string. The conversion factor identifier may allow for a translation or conversion of the medical data from a pure number into a true physical quantity. The conversion factor identifier may contain a representation of a unit of measurement and/or a numerical factor. The unit of measurement may be based on m (meter), kg (kilogram), s (seconds), min (minutes), h (hours), V (volts), A (ampere), cm³ (cubic centimeter), l (liter), hPa (hectopascal), mmHg (millimeter Mercury) or compound units or derived units thereof or the corresponding units of the imperial system of measurement. Alternatively the unit of measurement may be the international unit (I.U.) for doses of medicament(s), e.g. insulin. The unit of measurement may comprise a prefix to set a dimension of the unit of measurement, e.g. m (milli), μ (micro), n (nano), k (kilo), M (mega), G (giga), T (tera). The numerical factor may be 1 or any other value, e.g. a conversion factor between the respective unit of measurement in the SI unit system or the imperial unit system.

According to further embodiments the electronic system comprises at least one detector unit, wherein the electronic control unit is configured to acquire the medical data based on readings of the detector unit. Additionally or alternatively, the electronic system comprises an electromechanical interface to be coupled to a medical device, wherein the medical device may comprise one or more sensors to be coupled with the detector unit. The electronic system and the sensor unit may be separate units in electrical connection with each other or they may be a part of a single integral unit. The detector unit may comprise an electronic clock, e.g. a real time clock (RTC) and/or a counter counting specific time-intervals from a known point in time or since initialization. The detector unit may comprise at least one sensor for detecting or measuring a physical quantity, e.g. current, voltage, resistance, velocity, angular momentum, light intensity, magnetic flux, relative position (distance or angular distance/difference), chemical concentration or composition, pressure or similar.

The electronic system may further comprise a cell or battery unit, e.g. a rechargeable or non-rechargeable cell or battery unit, for powering the electronic control unit, the memory unit, the communication unit and/or the detector unit.

According to another embodiment, the medical data comprises at least a time information and/or a measurement value. The medical data may consist of only time information and one or more associated measurement values, e.g. a single measurement value, such as a value characterizing the size of a dispensed dose. The time information may be information stating a specific date and/or time or an amount of time passed from a known point in time or since initialization of the device. The measurement values may be values associated and/or calculated with sensor data from the detector unit or be that sensor data. The control unit may be configured to evaluate the sensor data and determine from a single sensor value or a series of sensor values the measurement value or measurement values. The time information may be the time information associated with the time at which the measurement data or the sensor data for determining the measurement data is collected.

The medical device may be a drug delivery or injection device. The medical data may be dose information, e.g. comprising an amount information (such as information on the dose of medicament dispensed by the device in one dispensing operation) and a time information (such as the time when the dispensing operation for dispensing the dose occurred). The dose information may an amount of medicament ejected from the medical device. The dose information may be determined from a distance that a plunger of the medical device moved in relation to a container for the medicament, e.g. a cartridge or a syringe. The distance the plunger moved may be detected from the plunger itself or from the movement, especially rotation, of any other part of a drive mechanism that drives the plunger. This also offers the option of a semi-automated treatment logging mechanism (dose diary).

According to further embodiments, the electronic control unit is configured to transmit non-medical data to the external device. The non-medical data may comprise a device identifier, especially of the medical device. Alternatively or additionally the non-medical data may comprise a current time value, a device type identifier, a patient identifier, a therapy identifier, a medicament identifier, and/or a conversion factor identifier.

The electronic control unit may be configured to transmit the data, e.g. the medical data or the non-medical data, upon receiving a request from the external device, especially only upon receiving a request. This may reduce power consumption and/or may allow the on-demand request by an external device.

Furthermore, a medical device, especially a needle based injection device and/or a pen-type device, is proposed, comprising an earlier described electronic system. Pen-type devices comprise a form similar to a pen, e.g. a fountain pen, thereby allowing an easy handling of the medical device by users such as for self-administration.

Also a medical device, especially a (needle based) injection device and/or pen-type device, is proposed, comprising an (electro-mechanical) interface configured to be coupled with such an electronic system. The medical device is expediently a drug delivery device. The electronic system may comprise a housing with an (electro-mechanical or purely mechanical) interface to couple with the medical device. The interfaces of the housing and the medical device may each comprise electrical contacts to electrically connect the electrical control unit and/or a detector unit of the electronic system with sensing means integrated in the medical device. Alternatively, the sensing means may be integrated into the electronic system. All electronic components may be incorporated into the electronic system. In such a modular system the electronic system may be reused after the medical device is replaced with a new one, e.g. after a stored medicament is used up, or the device is defective or reached an otherwise determined “end of usability”, thereby reducing costs. As an alternative for a medical device which can be releasably coupled with the electronic system, the electronic system may be an integral component of the medical device.

Alternatively the medical device may be a device for measuring blood glucose level, blood pressure, pulse rate, oxygen levels, body or skin temperature, electrocardiogram or other medical data. The medical device and/or the electronic system may be configured to perform the measurement (acquiring of the medical data) in a regular but infrequent manner, e.g. a few times per day.

The (needle based) injection device may comprise a drive mechanism including an energy buffer. The energy buffer may be pre-charged or may be charged upon setting a dose, such as by the user. The energy buffer may be a spring, e.g. a torsion spring or a compression spring, a compartment of compressed gas, e.g. air, an electrical cell or battery for driving an electrical motor, and/or other suitable means. Alternatively, the device may be a manually driven, e.g. user-driven, device.

The medical device may comprise at least one container filled with medicament or a receptacle for receiving such a container. The container may comprise medicament in an amount sufficient for a plurality of doses to be dispensed by the device.

In case the electronic system is integrated into the device and in case the device is a drug delivery device, the device can preferably be used with a plurality of containers. In other words, the device may be a reusable device. Of course, the electronic system as an add-on module could also be used with a reusable medical device. The user may just have to replace an emptied container in the receptacle with a new one and re-connect the receptacle to a drive mechanism of the device.

The medical device may be a variable dose device. In a variable dose device, the size of the dose of medicament to be delivered can be set within limits defined by the mechanism of the device.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F (ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Preferably in combination with the earlier described electronic system as a counterpart for the electronic device but not limited thereto an external device is proposed, the external device comprising at least one further electronic control unit configured to receive medical data from the earlier described electronic system. The further electronic control unit may comprise a memory unit for storing the received medical data. The further electronic control unit may comprise a processing unit for executing machine-readable code that is saved in the memory unit. The machine-readable code may be stored in a read-only memory of the memory unit. The machine-readable code may be stored in a rewritable memory to allow for updates. Alternatively, especially when the external device is a specific purpose/dedicated device, the further electronic control unit may comprise an application-specific integrated circuit (ASIC) instead of a processing unit for executing machine-readable code.

The further electronic control unit may be configured to send a data request to the electronic system.

The data request may be a request for non-medical data. The non-medical data may comprise a device identifier, especially of the medical device. Alternatively or additionally the non-medical data may comprise a current time value, a device type identifier, a patient identifier, a therapy identifier, a medicament identifier, and/or a conversion factor identifier. This may enhance device functionality and safety.

The data request may be a request for recorded medical data. The data request may comprise a range information indicative for at least one storage position of the medical data in the electronic system and wherein the further electronic control unit is configured to receive the medical data for the requested range and store the medical data. The range information may include a first identifier indicative of a first requested set of medical data. The range information may include a second identifier indicative of a last requested set of medical data. The range information may include the first and second identifier even when they are identical. The electronic control unit of the electronic system may be configured to transmit the requested recorded sets of medical data according to the requested range. The further electronic control unit may be configured to receive the requested sets of medical data.

According to a further embodiment, the further electronic control unit is configured to receive a data integrity value in correlation with the received medical data from the electronic system and the further electronic control unit is configured to calculate a validation value based on the medical data and at least one additional data that was not transmitted in correlation with the medical data and to compare the validation value with the data integrity value. The further electronic control unit is configured to calculate the validation value according to the same method as was used in the electronic system for calculating the data integrity value.

The additional data may be data already known to the external device or data that can be estimated by the further electronic control unit. The additional data may be at least one of a storage position identifier of the medical data in the electronic system, a device identifier, a device type identifier, a patient identifier, a therapy identifier, a medicament identifier, or a conversion factor identifier.

The storage position identifier may be estimated to be the next free storage position according to the data previously received in the external device. For example, the external device may already have received and/or stored N sets of medical data. The expected storage position identifier would therefore be the one for N+1.

The device identifier, the device type identifier, the patient identifier, the therapy identifier, the medicament identifier, or the conversion factor identifier may be data already known to the device from a previous transmission of data, e.g. during a set-up or pairing process of the electronic system and the external device.

According to further embodiments, when the data integrity value and the validation value do not match, the further electronic control unit is configured to estimate a corrected value for the additional data and repeat the calculation and comparison of the validation value based on the corrected value. The further electronic control unit may be configured to repeat the correction, calculation and comparison till the validation value matches the data integrity value. The further electronic control unit may be configured to repeat the correction, calculation and comparison till a set maximum of repetitions is reached. The number of repetitions may depend on or be the number of bits of the data integrity value. When the additional data comprises the storage position identifier, the further electronic control unit may be configured to increase the storage position identifier by one position (e.g. from N+1 to N+2) and repeat the calculation.

Alternatively, when no corrected value can be found with matching validation value and data integrity value, the electrical control unit may be configured to perform one or a couple of actions. One of these actions may be to request a retransmission of the data from the electronic system to rule out a transmission error. Another one of these actions may be to inform the user of the non-matching transmission. Especially when the external device is used by multiple users in connection with different medical devices or even the same medical device and there are different user profiles selectable on the medical device and/or the external device, and when a patient identifier and/or a device identifier is part of the additional data, it may be hinted to the user that a wrong user profile is selected.

The additional data may be an expected storage position identifier of the medical data in the electronic system and, when the validation value based on the corrected value matches the data integrity value, the further electronic control unit is configured to send the data request and the range information that lies between the corrected value and a last storage position information for which medical data was received, e.g. before the one that is currently evaluated.

The first identifier may therefore be the last storage position for which medical data was received or one higher. The second identifier may therefore be the corrected value or the value one below. The range information may include two position identifiers. The range information may include a storage position identifier for a first set of medical data missing in the storage of the further electronic control unit as first identifier. The range information may include a storage position identifier for a last set of medical data missing in the storage of the further electronic control unit as second identifier. The electronic system, e.g. the electronic control unit of the electronic system, may be configured to transmit the requested range of medical data sets upon receiving the request from the external device. The requested range may comprise more than two sets of medical data, e.g. three, four, five or more. Alternatively the further electronic control unit may send individual data requests for every data set missing in the storage of the further electronic control unit, e.g. even if there is more than one data set missing. Requesting the range may, however, be more efficient as regards power consumption, for example.

Furthermore, a corresponding method is proposed for exchanging data between an electronic system, e.g. an earlier described electronic system for a medical device, and an external device, e.g. an earlier described external device, the method comprising: establishing a communication channel between the electronic system and the external device and transmitting data, e.g. device data, from the medical device to the external device.

Prior to transmitting, the method may comprise the step of requesting specific data from the electronic system by the external device. Requesting specific data may include a data request for recorded medical data. The data request may comprise a range information indicative for at least one storage position of the medical data in the electronic system.

The electronic control unit may be configured to receive the medical data for the requested range and store the medical data.

The transmitting of data may further include transmitting medical data and at least one data integrity value in correlation with the medical data, the data integrity value being based on the medical data and at least one additional data that is not transmitted in correlation with the medical data.

The method may further include receiving the medical data and the data integrity value in the external device.

The method may additionally include calculating a validation value based on the received medical data and at least one additional data and comparing the validation value and the data integrity value.

The method may also include requesting additional medical data from the electronic system by the external device when the validation value does not match the data integrity value.

Machine-readable code is proposed that when executed in a control unit of an external device, e.g. in its processing unit, causes the external device to function as an earlier described external device. This may be an app on a user's/patient's smartphone. The machine-readable code may be stored on a data storage medium, e.g. in cloud storage, on the external device or distribution media like USB-Storage Sticks or CD's and the like.

Machine-readable code is proposed that when executed in a control unit of an electronic system for a medical device causes the electronic system to function as an earlier described electronic system. The machine-readable code may be stored on a data storage medium, e.g. in cloud storage or distribution media like USB-Storage Sticks or CD's and the like or provided by a data stream.

Further aspects, embodiments and advantages will become apparent from the following description of the exemplary embodiments in conjunction with the drawings.

### BRIEF DESCRIPTION OF THE DRAWINGS

FIG. 1 is a schematic view of a medical device comprising an electronic system and an external device.

FIG. 2 is a schematic view of an alternative medical device and an electronic system and an external device.

FIGS. 3a to 3b are a schematic view of a method for exchanging and evaluating data between an electronic system and an external device.

### DESCRIPTION OF EXEMPLARY EMBODIMENTS

In the figures, identical elements, identically acting elements or elements of the same kind may be provided with the same reference numerals.

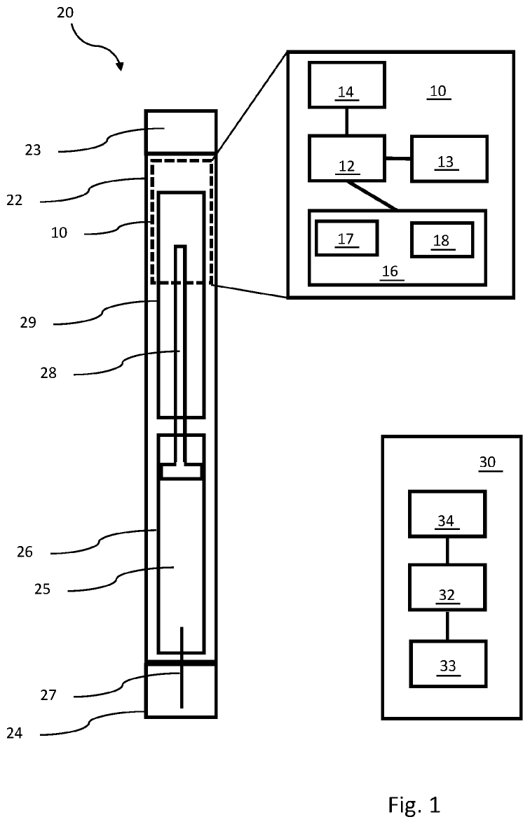

FIG. 1 shows a medical device 20. The medical device 20 comprises an electronic system 10 for the medical device 20 and for recording medical data, comprising an electronic control unit 12 configured to transmit the medical data to an external device 30 and store the medical data.

The electronic system 10 comprises a memory unit 13 for storing the medical data. The memory unit is electrically connected to electrical control unit 12. The electronic system 10 comprises a communication unit 14 for transmitting the medical data to the external device 30. The communication unit 14 is electrically connected with the electronic control unit. The communication unit 14 is a Bluetooth Low-Energy module. The electronic control unit 12 is an ASIC or a processing unit for executing machine-readable code.

The medical device 20 is a needle-based injection device (also called NIS—needle based injection system—in the following). The medical device 20 is a pen-type device. However, the present invention may also be implemented in other medical devices, e.g. in autoinjectors. The medical device 20 comprises an elongated housing 22. The medical device 20 comprises a receptacle 26 for receiving a container 25. The container 25 is filled with a medicament. The container 25 may be changed for a different one by partly disassembling the medical device, e.g. an empty container may be changed for a full one. The medical device 20 comprises a needle 27 at a first end that is fluidly connected with the container 25. The medical device 20 comprises a cap 24 that may be mechanically connected to the housing 22 and that is covering the needle 27 when connected with the housing 22. The medical device 20 comprises a dial 23 rotatably connected to the housing 22 at a second end opposite from the needle. A dose of medicament to be ejected from the medical device (and injected into the human body) may be selected by rotating the dial 23. The dial 23 also functions as a release to initiate ejection of the medicament upon a press along the cylinder axis. This may cause an energy buffer to release the energy for driving the medicament delivery. The medical device 20 comprises a drive unit 29 and a plunger 28. The drive unit 29 drives the plunger 28 for a distance specific to the set dose into the container 25. As an alternative to a device with a separate energy source for the drive mechanism, the device may be manually driven, i.e. the force required to move a plunger in a container may be provided by the user.

The electronic system 10 comprises a detector unit 16. The detector unit 16 is electrically connected to the electronic control unit 12. The detector unit 16 comprises an electrical clock 18. The detector unit comprises a movement detector 17 for detecting an amount of movement of the plunger 28. For driving the plunger 28, the drive unit 29 creates a rotary movement that is translated into a linear movement of the plunger 28. The movement detector 17 is provided to measure an amount of rotation in the drive unit to determine the amount of linear movement of the plunger 28. The movement detector 17 may work on an optical, electrical and/or magnetic basis. Alternatively, the movement detector might directly measure the linear movement of the plunger 28, e.g. by laser distance measuring or via a mechanical Vernier scale. The electronic control unit 12 is configured to acquire the medical data based on readings of the detector unit. The electronic control 12 unit is configured to determine a dose amount based on the readings of the movement detector 17 and associate a time value given by the electronic clock 18 with the dose amount to form a set of medical data. According to a different embodiment, the detector unit 16 may additionally or alternatively be directly connected to the memory unit 13 to store measured data directly while the electronic control unit 12 may be in a low energy/sleep state and the electronic control unit 12 may be configured to use data from the memory to determine the set of medical data.

The electronic control unit 12 is configured to store the medical data to the memory unit 13.

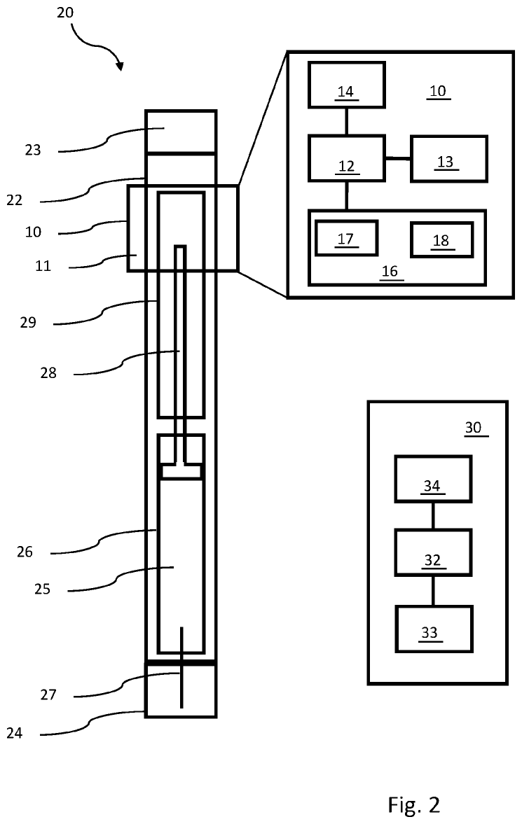

FIG. 2 shows an alternative medical device 20 and an electronic system 10. The medical device 20 does not comprise an electronic system. The electronic system 10 comprises a separate housing 11. The separate housing 11 and a housing 22 of the medical device 20 each comprise a mechanical interface with a locking feature to removably fasten the separate housing 11 to the housing 22. The separate housing 11 has a ring-like form so it can be mounted/pushed onto the housing 22. Alternatively other forms, e.g. a C-like form, are also possible. The electronic system 10 and the medical device 20 form a modular system. This allows the electronic system 10 to be used with multiple medical devices in alternating or serial fashion.

A drive unit 29 of the medical device 20 comprises markings, e.g. optic, magnetic and/or ferroelectric markings, that may be observed from outside the housing 22 by a motion detector 17 of the electronic system 10. According to alternative embodiments, the motion detector 17 may in part be integrated into the medical device 20 and an interface between the housing 22 and the separate housing 11 may comprise electrical and/or mechanical connections for the motion detector 17 to the detector unit 16. Alternatively the motion detector 17 may comprise a needle reaching into the housing 22 to mechanically scan ripples on a moving part of the drive unit 29. The needle may be formed from two parts—one part integrated into the housing 22 and one part integrated into the separate housing 11—that are mechanically or magnetically coupled.

Also shown in FIGS. 1 and 2 is the external device 30. The external device 30 is a smartphone. The external device 30 comprises a further electronic control unit 32. The further electronic control unit 32 comprises a processing unit executing specific machine-readable code. The machine-readable code is provided in the form of an app from a cloud storage (app shop). The further electronic control 32 unit is configured to receive medical data from the electronic system 10. The external device 30 comprises a communication unit 34 for receiving data from the electronic system 10. The communication unit 34 of the external device 30 is compatible with the communication 14 unit of the electronic system 10. The communication unit 34 is a Bluetooth module. The external device 30 comprises a memory unit 33 for storing the medical data.

In a first method, a communication channel is established between the external device 30 and the electronic system 10 for the first time and an initiation is performed. During initiation the external device 30 requests essential non-medical data from the electronic system 10. The further electronic device 32 is configured to send a data request for non-medical data to the electronic system 10. The electronic system 10 provides the non-medical data to the external device. The electronic control unit 12 is configured to transmit the non-medical data to the external device 30 upon request. The non-medical data comprises device-specific data (device data). The non-medical data includes a device identifier (UDI-unique device identification).

The non-medical data includes a current time signal of the electronic clock 18. The non-medical data includes a medicament identifier of the medicament in the container. The medicament type may be predefined in the settings of the electronic system 10 and only a specific medicament type may be fit into the medical device 20, or the detector unit 17 may comprise an additional sensor for detecting a container identifier, e.g. a specific color or form. The non-medical data includes a conversion factor identifier. The user may create a user profile in the external device. The further electronic control unit 32 may be configured to send a user identifier to the electronic system to be stored as non-medical data.

The electronic control 12 unit may be configured to transmit the non-medical data as a single string which simplifies the interface and minimizes the number of required commands. The external device 30, especially the further electronic control unit 32 may then use this information to do one or more of, but not limited to, the following actions:
Identify each unique medical device 20 (pen) that it is connected with.
Show UDI information to the user (serial number, lot, etc.).
Adapt its artwork based on color information about the medical device in the non-medical data.
Perform automated checks on memory capacity, production date and generate associated warning messages to the patient.
Provide the data partially to a cloud database or service center.

The further electronic control unit 32 may also send a data request for any selection of the non-medical data to the electronic system 10 at any time after the initiation when needed.

In other words: Connected NIS devices have an established data communication channel to transmit dose records to the external device. This data channel can be used or extended (e.g. by additional Bluetooth characteristics) to also transmit supplementary data. As this data is usually not required with every data transmission, a return channel to send request commands on demand has to be used or needs to be implemented, if not part of the basic data channel.

According to a second method, the electronic control unit 12 is configured to calculate a data integrity value based on the medical data and at least one additional data not included in the medical data and the electronic control unit is configured to store the data integrity value in correlation with the medical data. The data integrity value is calculated according to an 8 bit cyclic redundancy check (CRC) method. The data integrity value is an 8 bit value. The electronic control unit 12 is configured to regularly, e.g. on a daily, weekly or monthly basis, perform a data validation of the recorded medical data. The electronic control unit 12 is configured to recalculate the data integrity values based on the stored sets of medical data and the additional data and compare it with the stored data integrity values. When a difference is found in this comparison, the electronic control unit 12 may be configured to mark the faulty set of medical data as such or perform a correction operation based on the found difference.

To ensure that no record (set of medical data) is lost, each record gets a consecutive dose number, counting upwards from 0, with an increment of 1 (i.e. record numbers 0, 1, 2, 3, 4, etc.) Each record also contains an 8-bit cyclic redundancy check (CRC) type control value (data integrity value) for data integrity. The dose number is included when the CRC value is calculated, but not stored in the dose record itself. Instead, the device (medical device 20/electronic control unit 10) keeps a count of how many records are already stored in memory 13 and uses memory addressing to translate a dose record number into a specific memory address to select the correct dose record.

A third method, a method for exchanging data between the electronic system 10 and the external device 30 comprises sending a data request from the external device to the electronic system 10. The data request is for recorded medical data, wherein the data request comprises a range information indicative for at least one storage position of the medical data in the electronic system. The range information includes a first identifier for a storage position of a first requested set of medical data. The range information includes a second identifier for a storage position of a last requested set of medical data. The further electronic control 32 unit is configured to send a data request to the electronic system 10. The third method further comprises establishing a communication channel between the electronic system 10 and the external device 30. The third method further comprises transmitting data from the electronic system 10 to the external device 30. The further electronic control 32 unit is configured to receive the medical data for the requested range and store the medical data.

In other words: When the external device 30 is exchanged, the app reinstalled or a dose record transmission from the NIS 20 is interrupted or missed, the external device 30 may need to request the transmission of one or more dose records (sets of medical data) stored in the NIS's memory 13. To do so, the external device 30 sends a request command to the NIS 20. This may be achieved with one or more dose record identifiers (usually dose record numbers-storage position identifiers) to request a range of records or a single record. In the case of a consecutive range, two identifiers may be sent to identify the start and end parameters. In the case of a single record, either a single identifier, or two identifiers equal to one another, or two identifiers not equal to each other (in the case of non-inclusive implementation) may be sent. In order to minimize the consumption of internal memory, the NIS 20 does not store the dose record identifier explicitly within the dose record (medical data) itself, as it is usually given by the absolute dose record position in the internal memory 13. The NIS 20 may transmit the requested medical data together with a data integrity value calculated on basis of the medical data and the record identifier (storage position identifier). By transiently including the record identifier in the creation of a checksum (data integrity value), the identifier is implicitly stored within the record without any memory penalty. The external device 30 can then verify correctness of the dose record identity by adding the estimated and expected record identifier for checksum calculation and verification, the external device 30 may verify that the received records actually are the records previously requested. Dose records may contain an explicit checksum portion to confirm data integrity.

According to a fourth method, a method for exchanging data between the electronic system and the external device, the electronic system 10 and the external device 30 establish a connection. The electronic control unit 12 is configured to calculate a data integrity value based on the most recent medical data and at least one additional data not included in the most recent medical data and the electronic control unit 12 is configured to transmit the data integrity value to the external device 30 in correlation with the most recent medical data. The transmitting of data includes transmitting medical data and at least one data integrity value in correlation with the medical data, the data integrity value being based on the medical data and at least one additional data that is not transmitted in correlation with the medical data. The electronic control unit 12 is configured to not transmit the additional data in correlation with the medical data. The electronic control unit 12 is configured to never explicitly send the storage position data of the transmitted medical data.

The additional data includes a storage position identifier of the medical data. The additional data also includes the device identifier. According to other embodiments, other parameters may be included in or excluded from the additional data.

The further electronic control unit 32 is configured to receive the data integrity value in correlation with the received medical data from the electronic system 10. The medical data and the data integrity value are received in the external device. The further electronic control unit 32 is configured to calculate a validation value based on the received medical data and at least one additional data that was not transmitted in correlation with the medical data and to compare the validation value with the data integrity value. The further control unit 32 is configured to use a value stored in the memory unit 33 of the external device for part of the additional data (the device identifier). The further control unit 32 is configured to estimate another (the other) part of the additional data. The further control unit 32 is configured to estimate the storage position identifier to be one higher than a storage position identifier of the last received transmission before the currently received one.

In other words: In each initial and/or subsequent communication event between the NIS device and receptor, the current dose number is exchanged. From that time on, both devices independently count the number of records without directly exchanging the number again. However, it is vital that the devices stay synchronized. When new records are transmitted (usually in the order of time), the receptor device can use its own counter to estimate the dose numbers of the new records and include it in the CRC check. If it is successful (i.e. matches the CRC in the record), both data integrity and correct dose numbering are verified.

When the data integrity value and the validation value do not match, the further electronic control 32 unit is configured to estimate a corrected value for the storage position identifier and repeat the calculation and comparison of the validation value based on the corrected value. The storage position identifier is increased and the validation value recalculated until the validation value and the data integrity information match or a limit for increasing the storage position is reached.

When a corrected value for the storage position identifier is found for which the validation value based on the corrected value matches the data integrity value, the third method is executed to request additional medical data from the electronic system 10 by the external device 30, wherein the range information (e.g. for the requested medical data to be transmitted from the system to the device) lies between the corrected value and a last storage position information for which medical data was received.

In special cases, e.g. after the app is freshly (re) installed on the external device 30, the first identifier of the range information may be associated with the first data set recorded and/or stored in the electronic system 10.

In other cases the fourth method will trigger when the transmission of a previous record was not completed properly or when multiple sets of medical data were recorded since the last connection.

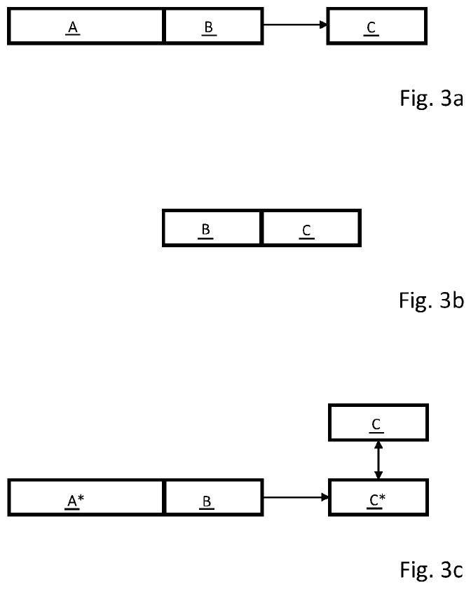

In FIG. 3a medical data B and additional data A is used to calculate a data integrity value C.

In FIG. 3b, only the medical data B and the data integrity value C are stored or transmitted in correlation. Redundant or easily guessed information A is neither stored nor transmitted in correlation with the medical data B or the data integrity value C.

In FIG. 3c, estimated additional data A* is used together with the received and/or stored medical data B to calculate a validation value C*. The validation value C* is then compared to the stored and/or received data integrity value C.

The scope of protection is not limited to the examples given herein above. Any invention is embodied in each novel characteristic and each combination of characteristics, which particularly includes every combination of any features which are stated in the claims, even if this feature or this combination of features is not explicitly stated in the claims or in the examples. The features of the embodiments mentioned above may be combined. The layout, function, and number of components may be changed in other embodiments.

### REFERENCE NUMERALS

10 Electronic system
11 Separate housing
12 Electronic control unit
13 Memory unit
14 Communication unit
16 Detector unit
17 Movement detector
18 Electronic clock
20 Medical device
22 Housing
23 Dial
24 Cap
25 Container
26 Receptacle
27 Needle
28 Plunger
29 Drive unit
30 External device
32 Further electronic control unit
33 Memory unit
34 Communication Unit
A Additional data
A* Estimated additional data
B Medical data
C Data integrity value
C* Validation value

1. Electronic system (10) for a medical device (20) and for recording medical data (B), comprising an electronic control unit (12) configured to transmit the medical data to an external device (30) and/or to store the medical data, wherein the electronic control unit (12) is configured to calculate a data integrity value (C) based on the medical data (B) and at least one additional data (A) not included in the medical data and wherein the electronic control unit is configured to, in correlation with the medical data, transmit the data integrity value to the external device and/or store the data integrity value.

2. Electronic system according to claim 2, wherein the electronic control unit (12) is configured to not transmit the additional data (A) in correlation with the medical data (B).

3. Electronic system according to any one of the preceding claims, wherein the medical data (B) comprises at least a time information and/or a measurement value, and/or wherein the medical device (20) is an injection device and the medical data is dose information.

4. Electronic system according to any one of the preceding claims, wherein the electronic control unit (12) is configured to transmit non-medical data to the external device (30).

5. Medical device, especially a needle based injection device and/or a pen-type device, comprising an electronic system (10) according to any of the preceding claims or comprising an interface configured to be coupled with such an electronic system.

6. Medical device according to claim 5, comprising at least one container filled with medicament or a receptacle for receiving such a container.

7. External device (30) comprising at least one further electronic control unit (32) configured to receive medical data (B) from an electronic system according to any of the claims 1 to 4, wherein the further electronic control unit is configured to receive a data integrity value (C) in correlation with the received medical data (B) from the electronic system (10) and wherein the further electronic control unit is configured to calculate a validation value (C*) based on the medical data and at least one additional data that was not transmitted in correlation with the medical data and to compare the validation value with the data integrity value, and wherein,
when the data integrity value and the validation value do not match, the further electronic control unit (32) is configured to estimate a corrected value for the additional data and to repeat the calculation and comparison of the validation value based on the corrected value.

8. External device according to claim 7, wherein the further electronic control unit (32) is configured to send a data request to the electronic system.

9. External device according to claim 8, wherein the data request is for recorded medical data (B), wherein the data request comprises a range information indicative for at least one storage position of the medical data in the electronic system (10) and wherein the further electronic control unit (32) is configured to receive the medical data for the requested range and store the medical data, and wherein
the additional data comprises an expected storage position identifier of the medical data in the electronic system and wherein, when the validation value based on the corrected value matches the data integrity value, the further electronic control unit is configured to send the data request and wherein the range information lies between the corrected value and a last storage position information for which medical data was received.

10. Method for exchanging data between an electronic system and an external device (30), the method comprising:
establishing a communication channel between the electronic system and the external device,
transmitting data, e.g. device data, from the electronic system (10) to the external device (30), wherein the transmitting of data includes transmitting medical data (B) and at least one data integrity value (C) in correlation with the medical data, the data integrity value being calculated by the electronic system based on the medical data and at least one additional data (A) that is not transmitted in correlation with the medical data,
receiving the medical data and the data integrity value in the external device,
calculating a validation value (C*) based on the received medical data and at least one additional data and comparing the validation value and the data integrity value,
estimating a corrected value for the additional data and repeating the calculation and comparison based on the corrected value, if the data integrity value and the validation value do not match.

11. The method of claim 10, wherein the electronic system is an electronic system (10) for a medical device (20) according to one of claims 1 to 4, and the external device is an external device (30) according to one of claims 7 to 9.

12. Machine-readable code that when executed in a control unit of an external device causes the external device to function as an external device according to any of the claims 7 to 9.

13. Data storage medium comprising the machine-readable code of claim 12.
